(19) United States
(12) Patent Application Publication
(10) Pub. No.: US 2005/0147582 A1
(43) Pub. Date: Jul. 7, 2005

(54) Package for cyanoacrylate adhesive

(76) Inventors: Robert Zimmerman, Rye, NY (US); Max Azevedo, Lenoir, NC (US)

Correspondence Address: WELSH & FLAXMAN LLC, 2450 CRYSTAL DRIVE, SUITE 112, ARLINGTON, VA, 22202, US

(21) Appl. No.: 11/029,386

(22) Filed: Jan. 6, 2005

Related U.S. Application Data
(60) Provisional application No. 60/534,360, filed on Jan. 6, 2004.

(51) Int. Cl. C09J 101/00; A61K 31/785

(52) U.S. Cl. 424/78.27; 156/331.8

(57) ABSTRACT

A cyanoacrylate delivery system includes a package body containing a cyanoacrylate adhesive composition. The package body is composed of an inner layer of nitrile polymer and an outer layer of polypropylene or aluminum.

### CROSS REFERENCE TO RELATED APPLICATIONS

This application is based upon U.S. Provisional Patent Application Ser. No. 60/534,360, entitled “PACKAGE FOR CYANOACRYLATE ADHESIVE”, filed Jan. 6, 2004.

### BACKGROUND OF THE INVENTION

1. Field of the Invention

The invention relates to packaging for cyanoacrylate adhesives. More particular, the invention relates to a nitrile polymer/polypropylene package for cyanoacrylate.

2. Description of the Prior Art

Medical interest in cyanoacrylate polymers has been apparent since at least the mid-nineteen sixties as evidenced by numerous reports on its use as a tissue bonding agent. Collins et al. reported on the effectiveness of homologous chain cyanoacrylates for bonding of biological substrates. J. A. Collins, et al. ARCH. SURG. Vol. 93, 428 September 1966; F. Leonard et al., J.A.P.S. Vol. 10: 1617, 1966. Both articles report the observation of higher rates of polymerization with longer chain esters than with the methyl or ethyl monomers. There appeared to be more biocompatibility with the longer chains as noted by the ease of spreading monomer films on bio-substrates. This contrasted with in vitro polymerizations where the lower homologues reacted much faster. There was particular interest in the degradation of these polymers as related to possible harmful effects that would preclude their use in surgery.

Throughout the development of medically useful cyanoacrylate adhesives, researchers have been confronted with problems relating to both the stability and application of the cyanoacrylate adhesives. Before these adhesives are commercially viable, the shelf-life of the stored adhesives must be sufficient to permit shipping and storage prior to us. In addition, the package must provide a convenient and reliable mechanism for the application of the cyanoacrylate adhesive. The present invention provides both a mechanism for increasing shelf-life and a convenient and reliable application structure.

### SUMMARY OF THE INVENTION

It is, therefore, an object of the present invention to provide a cyanoacrylate delivery system including a package body containing a cyanoacrylate adhesive composition, wherein the package body is composed of an inner layer of nitrile polymer and an outer layer of polypropylene.

It is also an object of the present invention to provide a delivery system wherein the package body has a bottle shape.

It is another object of the present invention to provide a delivery system wherein the package body includes a liquid containing area and a feed channel in fluid communication with the liquid containing area.

It is a further object of the present invention to provide a delivery system wherein the nitrile polymer is a copolymer composition.

It is still another object of the present invention to provide a delivery system wherein the copolymer composition is 2-propenoic acid, methyl ester, polymer with 1,3-butadiene and 2-propenenitrile.

It is yet another object of the present invention to provide a delivery system wherein the cyanoacrylate adhesive is octyl cyanoacrylate.

It is also another object of the present invention to provide a delivery system further including a middle layer between the nitrile polymer inner layer and the polypropylene outer layer.

It is a further object of the present invention to provide a cyanoacrylate delivery system including a package body containing a cyanoacrylate adhesive composition, wherein the package body is composed of an inner layer of nitrile polymer and an outer layer of aluminum.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a front view of a package in accordance with a first embodiment of the present invention.

FIG. 2 is a cross sectional view along the line II-II in FIG. 1.

FIG. 3 is a cross sectional view of the package body in accordance with an alternate embodiment of the present invention.

FIG. 4 is a cross sectional view of the package body in accordance with still a further embodiment of the present invention.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limited, but merely as the basis for the claims and as a basis for teaching one skilled in the art how to make and/or use the invention.

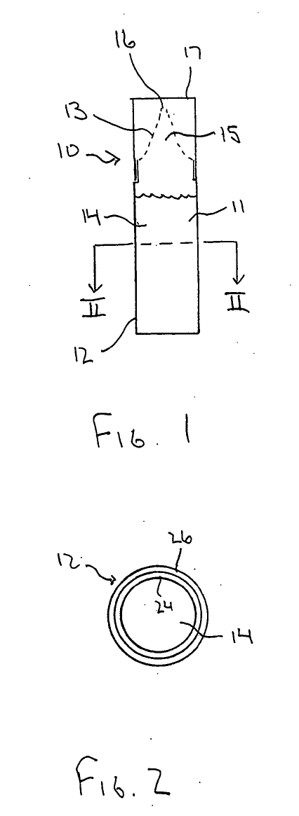

With reference to FIGS. 1 and 2, a package 10 for use in the transport and application of cyanoacrylate adhesive products 11 is disclosed. The package 10 is generally composed of a package body 12 and a cap 17. The package body 12 is generally constructed as a dispenser bottle and employs a distinct material composition composed of a nitrile polymer inner layer 24 defining an inner surface of the package body 12 and a polypropylene outer layer 26. This composition enhances the stability of the cyanoacrylate adhesive product 11 stored therein. With regard to the cap 17, it is preferably manufactured from polyolefins or nitriles, for example, polyethylene, polypropylene, polyester and nitrile polymer material (such as a copolymer composition, specifically, 2-propenoic acid, methyl ester, polymer with 1,3-butadiene and 2-propenenitrile, sold under the BAREX brand by BP Petrochemicals). In accordance with a preferred embodiment, the cyanoacrylate is octyl cyanoacrylate.

In accordance with the embodiment disclosed with reference to FIGS. 1 and 2, the package 10 includes a molded package body 12 having a centrally located liquid containing area 14 and a neck 13 extending therefrom. The cyanoacrylate adhesive product 11 is stored within the liquid containing area 14. The liquid containing area 14 is preferably sized to accommodate approximately 0.2 ml to approximately 2 ml of cyanoacrylate adhesive product as storage at this quantity is believed to provide ideal results. However, those skilled in the art will appreciate that other sizes could be employed without departing from the spirit of the present invention where specific applications require the use of more or less adhesive.

A feed channel 15 extends through the neck 13 and is in fluid communication with the liquid containing area 14. A spout 16 is positioned at the distal end of the neck 13 for the controlled dispensing of the cyanoacrylate adhesive and a cap 17 is provided for selectively covering the neck 13 and spout 16. As those skilled in the art will certainly appreciate, the cap 17 may be a screw-on type cap, a friction fit cap, or some other type of cap known within the industry. In addition, those skilled in the art will appreciate that a variety of package constructions may be employed without departing from the spirit of the present invention.

Regardless of the package construction employed in accordance with preferred embodiments of the present invention, improved cyanoacrylate stability is achieved by constructing the package body 12 from nitrile polymer making up the inner layer 24 of the package body. The nitrile polymer inner layer 24 provides a very durable package for the cyanoacrylate adhesive. In accordance with a preferred embodiment, the nitrile polymer material is a copolymer composition, specifically, 2-propenoic acid, methyl ester, polymer with 1,3-butadiene and 2-propenenitrile, sold under the BAREX brand by BP Petrochemicals.

BAREX offers high barrier properties which ensure the stability of the cyanoacrylate adhesive product stored therein. The exceptional barrier properties offered by BAREX make it an ideal inner layer material for use in construction of package bodies in accordance with the present invention. BAREX offers a high barrier to oxygen at all levels of relative humidity. This ensures that a consistently high barrier to oxygen is maintained, regardless of the humidity of the surrounding environment. In addition, the water vapor barrier properties of BAREX are comparable to other plastic packaging materials and are ultimately enhanced by the outer layer secured thereto in accordance with the present invention.

Referring to FIG. 2, a preferred embodiment of the package body 12 is composed of a multiple layer material formed in the shape of the package body 12. The multiple layer construction may be achieved via a variety of known manufacturing techniques, including, but not limited to, extrusion, blow molding, injection molding, thermoforming, flexible adhesive laminated substrate, etc.

Regardless of the manufacturing technique employed in accordance with the present invention, and in accordance with a preferred embodiment of the present invention, the package body 12 includes an inner layer 24 composed of nitrile polymer and an outer layer 26 composed of a thin layer of polypropylene, creating a package body 12 having a nitrile polymer inner layer 24 and a polypropylene outer layer 26.

The BAREX inner layer 24 has a thickness between approximately 0.001 mm to approximately 0.050 mm, preferably approximately 0.005 mm to approximately 0.015 mm, and the polypropylene outer layer 26 has a thickness between approximately 0.001 mm to approximately 0.050 mm, preferably approximately 0.001 mm to 0.005 mm. While preferred thicknesses are disclosed in accordance with the preferred embodiment, those skilled in the art will appreciate that the thickness have been chosen so as to provide a consumer durable item that maintains structural integrity while also permitting some degree of package body flexing so as to permit dispensing of the contents.

The polypropylene outer layer 26 creates a skin that attentuate moisture transmission into the package body 12. As such, those skilled in the art will appreciate that other olefinic moisture impervious outer films may be employed without departing from the spirit of the present invention. The combination of the stability offered by the nitrile polymer inner layer 24 and barrier of the polypropylene outer layer 26 provides a package body 12 offering a shelf life for cyanoacrylate adhesive products 11 stored therein of two or more years.

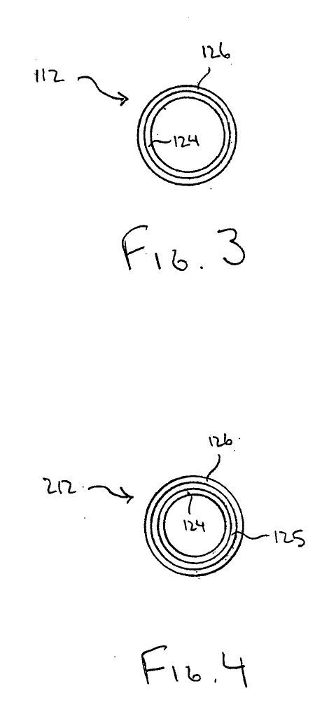

Although a BAREX/polypropylene multiple layer structure is employed in accordance with a preferred embodiment of the present invention, it is contemplated other multiple layer structures may be used within the spirit of the present invention. For example, and respectively with reference to FIGS. 3 and 4, it is contemplated the package body 112, 212 may be formed with an inner layer 124 of BAREX, and outer layer 126 of aluminum or and inner layer 224 of BAREX, a middle layer 225 of aluminum and an outer layer 226 of polypropylene. With regard to the thicknesses employed in accordance with these embodiments, the embodiment disclosed in FIG. 3 has a BAREX inner layer 124 that is between approximately 0.001 mm to approximately 0.050 mm, preferably approximately 0.005 mm to approximately 0.015 mm, and an aluminum outer layer 126 that is between approximately 0.001 mm to approximately 0.050 mm, preferably approximately 0.001 mm to 0.015 mm. The embodiment disclosed in FIG. 4 has a BAREX inner layer 224 that is between approximately 0.001 mm to approximately 0.050 mm, preferably approximately 0.005 mm to approximately 0.015 mm, an aluminum middle layer 125 that is between approximately 0.001 mm to approximately 0.050 mm, preferably approximately 0.001 mm to 0.005 mm, and a polypropylene outer layer 226 that is between approximately 0.001 mm to approximately 0.050 mm, preferably approximately 0.001 mm to 0.005 mm.

While the preferred embodiments have been shown and described, it will be understood that there is no intent to limit the invention by such disclosure, but rather, is intended to cover all modifications and alternate constructions falling within the spirit and scope of the invention.

1. A cyanoacrylate delivery system, comprising:
a package body containing a cyanoacrylate adhesive composition;
wherein the package body is composed of an inner layer of nitrile polymer and an outer layer of polypropylene.

2. The delivery system according to claim 1, wherein the package body has a bottle shape.

3. The delivery system according to claim 1, wherein the package body includes a liquid containing area and a feed channel in fluid communication with the liquid containing area.

4. The delivery system according to claim 1, wherein the nitrile polymer is a copolymer composition.

5. The delivery system according the claim 4, wherein the copolymer composition is 2-propenoic acid, methyl ester, polymer with 1,3-butadiene and 2-propenenitrile.

6. The delivery system according to claim 5, wherein the cyanoacrylate adhesive is octyl cyanoacrylate.

7. The delivery system according to claim 1, wherein the cyanoacrylate adhesive is octyl cyanoacrylate.

8. The delivery system according to claim 1, further including a middle layer between the nitrile polymer inner layer and the polypropylene outer layer.

9. The delivery system according to claim 8, wherein the nitrile polymer is a copolymer composition.

10. The delivery system according the claim 9, wherein the copolymer composition is 2-propenoic acid, methyl ester, polymer with 1,3-butadiene and 2-propenenitrile.

11. The delivery system according to claim 9, wherein the cyanoacrylate adhesive is octyl cyanoacrylate.

12. A cyanoacrylate delivery system, comprising:
a package body containing a cyanoacrylate adhesive composition;
wherein the package body is composed of an inner layer of nitrile polymer and an outer layer of aluminum.

13. The delivery system according to claim 12, wherein the package body has a bottle shape.

14. The delivery system according to claim 12, wherein the package body includes a liquid containing area and a feed channel in fluid communication with the liquid containing area.

15. The delivery system according to claim 12, wherein the nitrile polymer is a copolymer composition.

16. The delivery system according the claim 15, wherein the copolymer composition is 2-propenoic acid, methyl ester, polymer with 1,3-butadiene and 2-propenenitrile.

17. The delivery system according to claim 16, wherein the cyanoacrylate adhesive is octyl cyanoacrylate.

18. The delivery system according to claim 12, wherein the cyanoacrylate adhesive is octyl cyanoacrylate.
